(19) United States
(12) Patent Application Publication
(10) Pub. No.: US 2026/0108436 A1
(43) Pub. Date: Apr. 23, 2026

(54) SUNSCREEN COMPOSITION WITH SPF BOOSTERS

(71) Applicant: SAES GETTERS S.P.A., Lainate MI (IT)

(72) Inventors: Paolo VACCA, Milano MI (IT); Giorgio MACCHI, Gallarate VA (IT); Katarzyna FIDECKA, Cantù CO (IT); Stefano ZILIO, Bareggio MI (IT); Miriam RIVA, Lomazzo CO (IT); Simona PIROLA, Carugate MI (IT); Agnello DE SIMONE, Saronno VA (IT)

(73) Assignee: SAES GETTERS S.P.A., Lainate MI (IT)

(21) Appl. No.: 19/487,592

(22) PCT Filed: Apr. 4, 2025
(86) PCT No.: PCT/EP2025/059381

§ 371 Date: Nov. 25, 2025

(30) Foreign Application Priority Data
Apr. 5, 2024 (IT) 102024000007597
Apr. 5, 2024 (IT) 102024000007600
Jul. 22, 2024 (IT) 102024000016909
Jul. 22, 2024 (IT) 102024000016924

(51) Int. Cl. A61K 8/26; A61K 8/06; A61K 8/25; A61K 8/27; A61K 8/29; A61K 8/35; A61K 8/37; A61K 8/41; A61K 8/44; A61K 8/49; A61K 8/58; A61K 8/891; A61Q 17/04

(52) U.S. Cl. CPC A61K 8/26; A61K 8/062; A61K 8/25; A61K 8/27; A61K 8/29; A61K 8/35; A61K 8/37; A61K 8/41; A61K 8/416; A61K 8/445; A61K 8/4946; A61K 8/4966; A61K 8/585; A61K 8/891; A61Q 17/04

(57) ABSTRACT

The present invention relates to a sunscreen formulation with SPF booster based on microporous particles having a specific particle size distribution (PSD).

### FIELD OF INVENTION

The present invention relates generally to sunscreen compositions for protecting damage from ultraviolet (UV) rays. More specifically, the invention is direct to a formulation with SPF booster based on microporous particles having a specific particle size distribution (PSD).

### DESCRIPTION

The negative effects of exposure to ultraviolet (UV) light having a wavelength of from about 200 nm to about 400 nm, are generally recognized. The prolonged non-shielded exposure to solar radiation causes adverse health consequences, such as the immediately appearing of painful sunburns, and long-term damage which can lead to serious conditions such as skin cancer.

UV light also contributes to aging by causing free radicals to form in the skin. Free radicals include, for example, singlet oxygen, hydroxyl radical, the superoxide anion, nitric oxide and hydrogen radicals. Free radicals attack DNA, membrane lipids and proteins, generating carbon radicals. These in turn react with oxygen to produce a peroxyl radical that can attack adjacent fatty acids to generate new carbon radicals. This cascade leads to a chain reaction producing lipid peroxidation products. Damage to the cell membrane results in loss of cell permeability, increased intercellular ionic concentration, and decreased ability to excrete or detoxify waste products.

In order to reduce the amount of solar UV radiation received by the skin during exposure to the sun radiation, different sunscreen compositions can be used.

A sun care composition may contain inorganic UV filters, also called physical filters, and/or chemical UV filters which are organic molecules. The inorganic UV filters interact with UV light by two mechanisms: absorption and reflection/scattering, while the organic filters typically contain aromatic carbon and/or other electron-dense bonds that are responsible for absorbing light in the UV ranges of the solar spectrum.

The higher the amount of UV filters, the greater the degree of UV protection, however, it has recently become evident that too high concentration of both inorganic and organic UV filters, not only compromise the sun care formulation aesthetics, but it also brings unfavorable safety impacts on both human health and the environment.

Therefore, a key challenge that arose in this field is represented by the reduction of the quantities of UV filters used in sunscreen formulations while still ensuring high sun protection efficacy.

According to that, an effective strategy to solve this problem involves the use of a sun protection factor (SPF) booster into sunscreen formulations. SPF boosters are defined as compounds, safe for both human use and the environment, which are not recognized as sunscreen actives, but work to increase the SPF of the composition in which they are incorporated. According to some specific regulations (i.e. European Cosmetics Regulation 1223/2009), SPF formulations have to provide SPF protection consisting in UVA protection factor (UVAPF) at least on third of SPF with a critical wavelength of 370 nm or more. This is needed to reduce the UVA efficiency in penetrating the dermis and in promoting photosensitizing reaction able to generate harmful reactive oxygen species.

Typical sunscreen formulations are emulsions, serums, creams, or gels containing, sprays, in addition to chemical and/or physical UV filters, many other compounds such as emulsifiers, solubilizers, stabilizers, preservatives and also SPF boosters; all of which influence the protectiveness of the sunscreen, the activity of the SPF Booster and the and cosmetic appeal.

As reported in WO2017112982 and WO2021102873 a widely used inorganic SPF booster is represented by hydrophobic silica particles. Also, U.S. Pat. No. 9,265,715 claims for example the use of silica particles in combination with nylon and barium sulfate particles in order to have a boosting effect on sunscreen composition. A further example is represented by US20210330571 in which hydrophobic fine particulate of titanium oxide or zinc oxide, ensure a boosting effect to the final composition. Alternatively, WO2022081942 describes an organic compound, specifically xanthommatin, as SPF booster in combination with a chemical UV filter.

Another typical approach in the field of SPF booster is then represented by the use of particles characterized by specific dimensions which overlap the range of the UV light (i.e. about 200-400 nm), in order to exploit the UV scattering mechanism, as reported in U.S. Pat. No. 10,485,745.

However, some of the most cited classes of compounds such as silica has been progressively reduced due to their toxicity for human and environment. While inorganic compounds as titanium oxide and zinc oxide or organic compounds as the xanthommatin above reported, cannot be considered an SPF booster since they act as a real UV filter (the so-called SPF-doping effect).

Thus, object of the present invention is the development of a sunscreen composition characterized by the presence of a new versatile SPF booster.

In particular, the inventors found that in a sunscreen composition, comprising at least one UV filter, a surprisingly improvement of the booster effect on both UVB and UVA values can be obtained when the SPF booster is based on microporous particles having a particle size distribution (PSD) comprised between 100 nm and 10 μm, specifically divided into three fractions.

Said fractions are defined as a first fraction from 100 nm to 400 nm, a second fraction from 400 nm to 1 μm, and a third fraction from 1 to 10 μm, characterized by a ratio between the amount of the second fraction and the sum of the first and third fractions comprised between 0.1 and 5.

The zeolites particles size distribution (PSD) is measured using a state-of-the-art laser diffraction instrument. The powder is dispersed in DI water using an US bath up to a suitable concentration, to generate the optimum obscuration signal in the instrument. The slurry is recirculated from a container to the detector and the particle size distribution is measured. Particularly, the cumulative number distribution is evaluated.

The diameter fractions are calculated starting from the cumulative curve (e.g. the 0.1-0.4 μm fraction is the cumulative curve value in this range). All the SPF boosters of the present invention do not show a sunscreen activity as a single component, but act as an efficient SPF booster and UVA-PF booster when added in a formulation comprising UV filters; thus avoiding the SPF doper effect according to the international safety regulation.

According to the invention, the amount of SPF booster particles is comprised between 0.1 and 5% wt with respect to the total weight of the composition, while the amount of the UV filter is comprised 1.0 and 35% wt with respect to the total weight of the composition.

In a preferred embodiment, specifically when an organic UV filter is employed, the amount of said compound is comprised between 1 and 15% wt with respect to the total amount of the composition, while the amount is preferably comprised between 1 and 25% wt with respect to the total amount of the composition, when said filter is an inorganic compound.

Indeed, the UV filters employed in the sunscreen composition described herein can be an organic molecule, also defined as chemical filter, an inorganic compound, also defined as physical filter or mixture thereof.

Said organic molecule is selected in a group consisting of Diethylamino Hydroxybenzoyl Hexyl Benzoate, Ethylhexyl Salicylate, Bis-Ethylhexyloxyphenol Methoxyphenyl Triazine, Ethylhexyl Triazone, Phenylbenzimidazole Sulfonic Acid, Octocrylene, Homosalate, Butyl Methoxydibenzoylmethane, Homomenthyl Salicylate, ethylhexyl methoxycinnamate, Benzophenone-3, Ethylhexyl dimethyl para-aminobenzoic acid, Benzophenone-4, Trolamine Salicylate, Terephthalylidene Dicamphor Sulfonic Acid, Drometrizole Trisiloxane and 4-Methylbenzilidene Camphor polysilicone-16. Avobenzone, Diethylhexyl Butamido Triazone, Tris-biphenyl triazine, Tris-biphenyltrazine, Oxybenzone, Octyltriazone, Octisalate, Padimate O, Bis-piperazine HAA299, (2-Ethoxyethyl (2Z)-2-cyano-2-[3-(3-methoxypropylamino) cyclohex-2-en-1-ylidene]acetate), Sulisobenzone, Drometrizole trisiloxane, Methylene bis-benzotriazolyl tetramethylbutylphenol, Benzylidene camphor sulfonic acid, 4-Methylbenzylidene Camphor, Polysilicone-15, Sulisobenzone sodium, Aminobenzoic acid, PEG-25 PABA, Menthyl anthranilate, Isopentyl-4-methoxycinnamate, Dioxibenzone, Cinoxate, camphor benzalkonium sulfate and mixtures thereof.

While the inorganic compound is selected among Zinc Oxide, Titanium Dioxide, Aluminum Stearate, Alumina, Hydrated Silica or Aluminum Hydroxide, their mixtures, and surface modified oxides.

In a specific formulation, the UV filters can also include bacterial based systems.

In a preferred embodiment, the SPF booster particles are characterized by a crystalline structure with a crystallinity higher than 30% and an aspect ratio comprised between 0.1 and 1, where the aspect ratio is defined as the ratio between the width (W) and height (H) based on the bounding rectangle projection of the particle's shape in a 2D image.

According to a further aspect of the invention, said booster particles are characterized by a refractive index comprised between 1.35 and 1.65.

Both inorganic and organic materials can be employed as SPF booster characterized by the specific dimension distribution reported above.

Specifically, preferred inorganic particles are zeolites characterized by an orthorhombic or cubic cell structure and a surface area comprised between 200 and 1000 m²/g.

In a preferred embodiment said zeolites are selected among Faujasite type (FAU), Linde type A (LTA), Mordenite type (MOR), Beta type or a mixture of Linde type A (LTA) and ZSM-5 type.

While, preferred organic particles according to the invention are metal organic frameworks, preferably cyclodextrins, with a surface area comprised between 1000 and 5000 m²/g.

As reported in the below experimental part, the boost of the UVB performance ensured by said specific zeolites to the final composition is at least 20% and, at the same time, the UVA-PF boosting is at least 10%.

The sunscreen composition herein disclosed can be formulated as a one phase formulation which brings the advantages for the cosmetic aspect since it does not leave a greasy or white cast on skin, it is well suited to use under and over makeup and it does not clog skin pores. But it also ensures benefits for the sunscreen activity since it specifically targets certain areas that need increased protection like cheeks, nose or ears and it is typically the most water-resistant formula.

Thus, the single-phase formulation, preferably in the form of a stick, according to the present invention is a water-free phase selected in a group consisting of all types of oils, waxes, emollients, emulsifiers, silicones, polymers, butters, film formers, solubilizers, organic/inorganic dispersants and rheology modifiers, fats, solvents and carriers, stabilizers, organic and inorganic gellifying ingredients.

In a further embodiment, the same water free phases above cited, can be combined with a water phase in order to have an oil-in water formulation which is characterized by a high sensory and feeling on skin, due to a correct balance between the water and oil phases. Said homogenous oil-in-water emulsions are preferably formulated as a serum or cream.

The sunscreen composition according to the invention can also comprise at least one of a cosmetically acceptable emollient, carrier fluid, pigment, humectant, vitamin, antioxidant, natural colorant, preservatives, aroma ingredient, conditioner, emulsifiers, solubilizer, dispersant, film former, rheology modifier, sensory modifier, solvent, carrier, stabilizer, texturizer, filler, extract, essential oil or surfactant.

The present invention also refers to the composition according to any of the embodiment herein disclosed as a sunscreen composition or agent. In particular, the invention refers to a composition according to any of the embodiment herein disclosed for use in a method for protecting a keratinous surface (e.g. skin) from UV radiation comprising contacting the keratinous surface with the sunscreen composition of the invention.

The invention also refers to the composition as herein disclosed as an additive or adjuvant to a sunscreen composition.

Surprisingly, the SPF boosting effect is reached by the compositions and/or functionalized zeolites of the invention without the zeolite binding to the filter, e.g., with no absorption or encapsulation. According to the present invention, in fact, the zeolite and the UV filter do not interact but simply form a physical mixture. As demonstrated by SEM images (FESEM—EDS (Supra 55VP/Zeiss+UltraMax 170 mm2/Oxford Instruments)), according to the present invention, the zeolite is not integrated and is not part of the UV filter and does not act as a carrier. As will be apparent from the following experimental section, the zeolites according to the invention have rather a booster action. This is also confirmed by the fact that without a UV filter, zeolites of the invention have no action as a solar filter.

As will be apparent to a person skilled in the art, the composition of the invention can be included in diverse items and products such as: leave-on skin lotions and creams, shampoos, hair conditioners, shower gels, toilette bars, antiperspirants, deodorants, shave creams, lipsticks, lip-balms, stick formulations, foundations, sunscreen lotions/creams and the like. It is noted that sunscreen agents are active only when exposed to UV radiations. In cases where no prevention against UV radiations/sunburn is needed, for example when the composition is used as a shower gel, applying the composition to the skin cannot be considered as a therapeutic treatment and the composition is mostly used as a cosmetic rather than a medicament.

Therefore, the present invention also refers to non-therapeutic uses, e.g., cosmetic uses, of the composition, according to any one of the embodiments disclosed herein.

Other aspects and advantages of the invention will be apparent from the experimental section below.

### EXAMPLES

Hereinafter, the invention will be explained in more detail with reference to the following non-limiting examples. Modifications or variations of the embodiments here exemplified, obvious to an expert in the art, are encompassed by the appended claims.

### Preparation of Comparative and Inventive Sunscreen Compositions (P5, S1-S2)

Reference sunscreen formulation P5 as stated in ISO norm 24444:2019 “Cosmetics—Sun protection test Methods”, is used as base formulation for SPF booster and UVA-PF booster evaluation. As reported in Table 1 sunscreen reference formulation P5 contains a mixture of different chemical filters made of 3% wt of butyl methoxydibenzoylmethane, 10% wt of octocrylene, 5% wt ethylhexyl salicylate, 5% wt of benzophenone-3.

TABLE 1
P5 reference formulation

| Ingredients | % of total mass |
|---|---|
| Phase A1 | |
| Water | 39.35 |
| Disodium EDTA | 0.05 |
| Methylparaben | 0.35 |
| Chlorphenesin | 0.2 |
| Phenoxyethanol | 0.7 |
| Phase A2 | |
| Glycerin | 5 |
| Phase B1 | |
| Xanthan Gum | 0.01 |
| Butyl Methoxydibenzoylmethane | 3 |
| Octocrylene | 10 |
| ethylhexyl Salicylate | 5 |
| Benzophenone-3 | 5 |
| Phase B2 | |
| PPG-2 Myristyl Ether Propionate | 2 |
| Octyldodecyl Neopentanoate | 2 |
| Butyloctyl Salicylate | 8 |
| PVP/Eicosene Copolymer | 1.3 |
| Phase B3 | |
| Polyglyceryl-3 Methyl Glucose Distearate | 2 |
| Cetyl Alcohol | 0.5 |
| Stearic Acid | 1 |
| Butylparaben | 0.03 |
| Phase C | |
| Cyclopentasiloxane | 3 |
| Acrylates/C10-30 Alkyl Acrylates | 0.2 |
| Crosspolymer | |
| Phase D | |
| Water | 1 |
| Triethanolamine (99%) | 0.06 |
| Phase E | |
| Water | 10 |
| Potassium Cetyl Phosphate | 0.25 |

The final formulation of S1 and S2 is then prepared by transferring 97 g of P5 in a mixer jar and adding 3.0 grams of “SPF booster” under stirring. Then homogenizer is used to allow a fine particles distribution.

### Preparation of Stick Formulation-UV Filters—Formulation Placebo #B1

### Methodology:

1. Combine and mix together the reagents of phase A (1-5) and heat up at 80-85° C., until well melted and homogenous.

2. Separately, blend together all the reagents of phase B (6-9) and heat up at 80-85° C., until well melted and homogenous.

3. Add phase B to phase A and stir until homogenous.

4. Take it off the heat and mix with prior-blended phase C (10-12). Pour off while still molten.

TABLE 2

| Phase | Reagent | INCI | w/w % | Function |
|---|---|---|---|---|
| A. | | | | |
| 1. | Caprylic/capric | Caprylic/capric | 5 | Emolient |
| | triglicerides | triglicerides | | |
| 2. | Isoamyl laurate | Isoamyl laurate | 13 | Emolient |
| 3. | C12-C15 Alkyl | C12-C15 Alkyl | 8 | Emolient |
| | benzoate | benzoate | | |
| 4. | Oilkemia ™ 5S | Caprylic/capric | 40 | Dispersant / Rheology |
| | polymer, Lubrizol | triglicerides (and) | | Modifier / Sensory |
| | | polyurethane-79 | | Modifier / Suspending |
| | | | | Agent / Thickener |
| 5. | SPF Booster | | 0 | SPF Booster |
| B. | | | | |
| 7. | Parsol 340 ®, DSM | Octocrylene | 10 | UV filter |
| 8. | Parsol EHS ®, DSM | Ethylhexyl | 5 | UV filter |
| | | Salicylate | | |
| 9. | Parsol HMS ®, DSM | Homosalate | 10 | UV filter |
| | Parsol 1789 ®, DSM | Avobenzone | 3 | UV filter |
| C. | | | | |
| 10. | Carrot oil | Zea Mays Germ Oil, | 4 | Emolient, colorant |
| | | Daucus carota | | |
| | | sativa Root Extract | | |
| 11. | Glucate DO ™ | Methyl Glucose | 1 | Emollient / Emulsifier |
| | emulsifier, Lubrizol | Dioleate | | |
| 12. | Fragrance | | 1 | Parfum |

### Preparation of Stick Formulation-UV Filters and Zeolite SPF Booster Powder—Formulation Samples #S3-S4

### Methodology:

1. Combine and mix together the reagents of phase A (1-5) and heat up at 80-85° C., until well melted and homogenous.
2. Separately, blend together all the reagents of phase B (6-9) and heat up at 80-85° C., until well melted and homogenous.
3. Add phase B to phase A and stir until homogenous.
4. Take it off the heat and mix with prior-blended phase C (10-12).

Pour off while still molten.

TABLE 3

| | | | w/w % | w/w % | |
|---|---|---|---|---|---|
| Phase | Reagent | INCI | S3 | S4 | Function |
| A. | | | | | |
| 1. | Caprylic/capric | Caprylic/capric | 5 | 5 | Emolient |
| | triglicerides | triglicerides | | | |
| 2. | Isoamyl laurate | Isoamyl laurate | 13 | 13 | Emolient |
| 3. | C12-C15 Alkyl | C12-C15 Alkyl | 8 | 8 | Emolient |
| | benzoate | benzoate | | | |
| 4. | Oilkemia ™ 5S | Caprylic/capric | 39 | 39 | Dispersant / Rheology |
| | polymer, | triglicerides | | | Modifier / Sensory |
| | Lubrizol | (and) | | | Modifier / Suspending |
| | | polyurethane- | | | Agent / Thickener |
| | | 79 | | | |
| 5. | MOR zeolite | | 3 | — | SPF Booster |
| | BETA zeolite | | — | 3 | |
| B. | | | | | |
| 7. | Parsol 340 ®, | Octocrylene | 10 | 10 | UV filter |
| | DSM | | | | |
| 8. | Parsol EHS ®, | Ethylhexyl | 5 | 5 | UV filter |
| | DSM | Salicylate | | | |
| 9. | Parsol HMS ® | Homosalate | 10 | 10 | UV filter |
| | DSM | | | | |
| | Parsol 1789 ® | Avobenzone | 3 | 3 | UV filter |
| | DSM | | | | |
| C. | | | | | |
| 10. | Carrot oil | Zea Mays Germ | 4 | 4 | Emolient, colorant |
| | | Oil, Daucus | | | |
| | | carota sativa | | | |
| | | Root Extract | | | |
| 11. | Glucate DO ™ | Methyl Glucose | 1 | 1 | Emollient / Emulsifier |
| | emulsifier, | Dioleate | | | |
| | Lubrizol | | | | |
| 12. | Fragrance | | 0.5 | 0.5 | Parfum |

### Preparation of Oil in Water Cream Formulation-UV Filters and Zeolite SPF Booster Powder—Placebo B2 and Sample #S5

Zeolites were incorporated into a ready, marine-friendly solar emulsion through post-process addition. Specifically, a ready O/W emulsion with SPF 50 was mixed with BETA zeolites using a Turrax at 5000 r.p.m for 5 minutes at room temperature until homogenously blended.

TABLE 4

| | | % | % |
|---|---|---|---|
| | | W/W | W/W |
| Phase | INCI | P7 | S5 |
| Continueous | Aqua (Water) | 100 | 97 |
| phase | | | |
| UV filters | Diethylamino Hydroxybenzoyl Hexyl | | |
| | Benzoate-DHHB, | | |
| | Ethylhexyl Salicylate- EHS | | |
| | Ethylhexyl Triazone- EHT | | |
| | Bis-Ethylhexyloxyphenol Methoxyphenyl | | |
| | Triazine-BEMT | | |
| | Phenylbenzimidazole Sulfonic Acid-PBSA | | |
| Emulsifier | Polyglyceryl-6 Stearate | | |
| Other | C12-15 Alkyl Benzoate, Butyloctyl | | |
| reagents | Salicylate, Lactobacillus Ferment, | | |
| | Polyglyceryl-6 Stearate, Vp/Eicosene | | |
| | Copolymer, Methyl Glucose | | |
| | Sesquistearate, Potassium Cetyl | | |
| | Phosphate, Glycerin, Aminomethyl | | |
| | Propanol, Hydroxyacetophenone, | | |
| | Ethylhexylglycerin, Polyglyceryl-6 | | |
| | Behenate, Tocopheryl Acetate, Sodium | | |
| | Gluconate, O-Cymen-5-Ol, Diglycerin, | | |
| | Pinus Pinaster Extract (Pinus Pinaster | | |
| | Bark Extract), Sodium Hydroxide, | | |
| | Ethylhexylglycerin, Hydroxyacetophenone, | | |
| | O-Cymen-5-Ol | | |
| SPF Booster | BETA 23.8/NH4+ | — | — |
| | BETA 38/ NH4+ | — | — |
| | BETA307/H+ | — | 3 |

### In Vitro UVA Protection Factor and Critical Wavelength According to ISO 24443:2021 International Standard (for S1-S2, C1-C2 and P5)

The test is based on the assessment of UV-transmittance brought by a thin film of a sunscreen product spread on a roughened substrate by means of a spectrophotometric method. The Helioplate SB6 Sandblasted PMMA plate from HelioScreen Cosmetic Science SAS with the batch number 498 has been used. Standard quantity of 1.2 mg/cm² (±1.5%) of cosmetic formulation has been applied onto each substrate by using an automatic dispenser. The minimum application area and size was at least equal to 22.1 cm². Finally, the applied sunscreen product was submitted to a drying step for least 30 min and less than 60 min in the dark conditions.

Measurements were performed before and after UV exposure with a specific measured and controlled dose of UV radiation from a defined UV exposure source to take into account the potential photo-stability characteristics of the test product. For this test a pre-irradiation dose of 25 J/cm² was applied.

### In Vitro SPF (Sun Protection Factor) According to ISO/DIS 23675 (for S1-S2, C1-C2 and P5)

The test is based on the assessment of the UVB (Ultraviolet-B) protection of sunscreen products by means of a UV spectral absorbance curve from an in vitro procedure recommended by Cosmetics Europe [Annex I: Double Plate Method Protocol—COSMETICS EUROPE RECOMMENDATION N° 26 ON THE USE OF ALTERNATIVE METHODS TO ISO24444:2019] and under progress at the ISO level under the project ISO/DIS 23675 [ISO/DIS 23675-Cosmetics-Sun protection test Methods—In Vitro determination of Sun Protection Factor]. Results of this measurement procedure are used for calculation of Sun Protection Factor (SPF).

### SPF In-Vitro Evaluation (for S3-S7 and B1-B2)

It has been developed an internal full in-vitro method to calculate the protection factor (SPF_{in vitro}) of a sunprotection product against erythema-inducing radiation calculated with a calibration based on a set of sunprotection standards.

The method is formulated according to the principles recommended (Protocol named “In vitro SPF Double Plate method) by the European Cosmetics and Perfumery association (former Colipa, then Cosmetics Europe) in 2011 and internally modified.

The test is based on the assessment of UV-transmittance through a thin film of sunscreen sample spread on a roughened PMMA (Poly(methyl-methacrylate)) substrate. In the inspiring procedure, exposure to a radiation from an UV exposure source is required. UV exposure is not used in this method; each set of sunscreen transmission data is mathematically corrected so that the in vitro SPF data yield the same SPF values provided by several certified SPF standards.

A Perkin-Elmer LAMBDA 1050+UV/Vis/NIR spectrophotometer equipped with a 150 mm Perkin-Elmer LAMBDA integrating sphere module is used to measure the absorbance A properties of the sunscreen on the test plates and absorbance values are used to calculate SPF through the following integral formula:

$${SPF}_{in vitro} = \frac{{\int }_{290 nm}^{400 nm} E ( λ ) \times I ( λ ) d λ}{{\int }_{290 nm}^{400 nm} E ( λ ) \times I ( λ ) \times {10}^{- C \times A ( λ )} d λ}$$

### Where:

E: tabled CIE-1987 erythema action spectrum;
I: tabled Midday mid-summer global irradiance at 40° N;
A: measured absorbance;
C: correction coefficient

The UV spectrophotometer wavelength range shall span the primary waveband of 290 to 400 nm, covering both the ranges 290-320 nm (UV-B) and 320-400 nm (UV-A) required in SPF calculation.

A correction coefficient C such that SPF_{in vitro}=SPF_{i, standard}, i=P1, P2, P3 (certified standards in different ranges of sunprotection, see table herebelow for certified values) is used in the calculation.

Mean and Acceptance limits of SPF reference
formulations -Compliance ISO 24444: 2019

| formulation | mean | Lower Limit | Upper Limit |
|---|---|---|---|
| P2 | 16.1 | 13.7 | 18.5 |
| P5 | 30.6 | 23.7 | 37.4 |
| P8 | 63.1 | 43.9 | 82.3 |

In the following table it is reported a mean SPF boosting value (%), calculated as:

$$mean SPF boosting ( \% ) = \frac{[ ( {SPF}_{B} - {SPF}_{P} ) - ( {SPF}_{F} - {SPF}_{P} ) ]}{{SPF}_{F} - {SPF}_{P}} \times 100$$

### Where:

SPF_{B}: measured SPF of sample formulation with UV filter plus UV booster;
SPF_{F}: measured SPF of sample formulation with UV filter;
SPF_{P}: measured SPF of placebo formulation (without UV filter and UV booster)

TABLE 5

| | | | | | SPF | | UVA-PF |
|---|---|---|---|---|---|---|---|
| | SPF | Frame- | Fractions | | boosting | UVA- | boosting |
| Ref. | booster | work | ratio | SPF | (%) | PF | (%) |
| P5 | no | n.a. | n.a. | 39.1 | 0 | 15.1 | 0 |
| B1 | no | n.a. | n.a. | 26.0 | 0 | 11.0 | 0 |
| B2 | no | n.a. | n.a. | 59.7 | 0 | 24.0 | 0 |
| S1 | Zeolite | LTA + | 0.9 | 53.7 | 37 | 20.9 | 38 |
| | | ZSM-5 | | | | | |
| S2 | Zeolite | FAU | 2.1 | 48.3 | 24 | 17.0 | 13 |
| S3 | Zeolite | MOR | 0.5 | 56.2 | 113 | 20.1 | 83 |
| S4 | Zeolite | BETA | 1.6 | 52.6 | 99 | 18.3 | 66 |
| S5 | Zeolite | BETA | 1.6 | 136.1 | 128 | 44.6 | 86 |
| S6 | Zeolite | LTA | 1.4 | 93.1 | 56 | 35.0 | 46 |
| S7 | Zeolite | LTA | 1.0 | 101.3 | 70 | 31.0 | 29 |
| C1 | Zeolite | ZSM-5 | 5.1 | 45.6 | 16 | 16.2 | 7 |
| C2 | silica | n.a. | 0.01 | 50.9 | 30 | 16.5 | 9 |

Table 5, clearly shows that the presence of an SPF booster based on microporous particles having the specific particle size distribution (PSD) of the present invention contribute to a boost both the UVB and the UVA performances.

Indeed, when it is employed an SPF booster characterized by a ratio between the second fraction and the sum of the first and third fractions comprised between 0.1 and 5.0 (see samples S1-S7) the SPF booster is higher than 20% and, at the same time, the UVA-PF boosting is increase of at least 10%.

On the contrary, zeolite particles with a higher ratio (see C1) do not reach the required increased of both UVB and UVA values. At the same time, typical UV boosters such as the silica particles reported in counterexample C2 characterized by a ratio of 0.01, show that even if they are characterized by an SPF boosting effect of at least 30%, they are not able to significantly boost the UVA-PF values.

### SPF In-Vivo Evaluation

As a further confirmation of the effectiveness of the present invention, the formulation according to the present invention have also been tested in vivo to assess the SPF of sunscreen formulations according to ISO 24444:2019 standard method. Ten healthy male and females subjects having skin phototype I to III according Fitzpatrick are enrolled by a board certified dermatologist.

A section of each subject's skin is exposed to ultraviolet light without any protection and another (different) section is exposed after application of the sunscreen product to be tested. One further section is exposed after application of a SPF reference sunscreen formulation (used for validation). To determine the SPF, incremental series of delayed erythemal responses are induced on a number of small sub-sites on the skin. These responses are visually assessed for presence of redness 20±4 hours after UV exposure, by a trained technician.

The minimal erythemal dose (MED) for unprotected skin (MEDu) and the MED obtained after application of a sunscreen product (MEDp) are determined on the same subject on the same day. An individual sun protection factor (SPFi) for each subject tested is calculated as the ratio of individual MED on product protected skin divided by the individual MED on unprotected skin i.e., MEDp/MEDu. The sun protection factor for the product (SPF) is the arithmetic mean of all valid SPFi results from each subject in the test

The test is considered valid for the first ten subjects if the resulting range of the 95% CI of the mean SPF is within ±17% of the mean SPF. If it is not within ±17% of the mean SPF, the number of subjects is increased stepwise from the minimum number of 10 until the 95% CI statistical criterion is met.

TABLE 6
In vivo results

| | Ingredient as | | SPF value ± | % SPF |
|---|---|---|---|---|
| Ref. | SPF booster | Formulation | StDev | boosting |
| B1 | none | Stick - water free | 30.1 ± 2.4 | Negligible |
| B2 | none | Serum - oil in water | 18.5 ± 3.1 | Negligible |
| S8 | FAU zeolite | Stick - water free | 37.2 ± 4.1 | 25.54 |

1. A sunscreen composition comprising:
a) at least one UV filter; and
b) at least an SPF booster based on microporous particles having a particle size distribution (PSD) ranging between 100 nm and 10 μm, said PSD including a first fraction from 100 nm to 400 nm,
a second fraction from 400 nm to 1 μm, and
a third fraction from 1 to 10 μm,
wherein a ratio of an amount of the second fraction to a sum of the first and third fractions ranges between 0.1 and 5.

2. The composition according to claim 1, wherein an amount of the SPF booster particles ranges between 0.1 and 5 wt % with respect to a total weight of the composition.

3. The composition according to claim 1, wherein an amount of the UV filter ranges between 1.0 and 35 wt % with respect to a total weight of the composition.

4. The composition according to claim 1, wherein the SPF booster particles are characterized by a crystalline structure with a crystallinity higher than 30%.

5. The composition according to claim 1, wherein the SPF booster particles are characterized by an aspect ratio ranging between 0.1 and 1.

6. The composition according to claim 1, wherein the SPF booster particles are characterized by a refractive index ranging between 1.35 and 1.65.

7. The composition according to claim 1, wherein the SPF booster particles are made of an inorganic material with a surface area ranging between 200 and 1000 m²/g.

8. The composition according to claim 7, wherein the inorganic particles are zeolites characterized by orthorhombic or cubic cell structure.

9. The composition according to claim 8, wherein the zeolites are Faujasite type (FAU) zeolites, Linde type A (LTA) zeolites, Mordenite type (MOR) zeolites, Beta type zeolites, or a mixture of Linde type A (LTA) and ZSM-5 type zeolites.

10. The composition according to claim 1, wherein the SPF booster particles are made of an organic material with a surface area ranging between 1000 and 5000 m²/g.

11. The composition according to claim 10, wherein the organic particles are metal organic frameworks.

12. The composition according to claim 1, wherein the UV filter is an organic molecule selected from the group consisting of Diethylamino Hydroxybenzoyl Hexyl Benzoate, Ethylhexyl Salicylate, Bis-Ethylhexyloxyphenol Methoxyphenyl Triazine, Ethylhexyl Triazone, Phenylbenzimidazole Sulfonic Acid, Octocrylene, Homosalate, Butyl Methoxydibenzoylmethane, Homomenthyl Salicylate, ethylhexyl methoxycinnamate, Benzophenone-3, Ethylhexyl dimethyl para-aminobenzoic acid, Benzophenone-4, Trolamine Salicylate, Terephthalylidene Dicamphor Sulfonic Acid, Drometrizole Trisiloxane, 4-Methylbenzilidene Camphor polysilicone-16, Avobenzone, Diethylhexyl Butamido Triazone, Tris-biphenyl triazine, Tris-biphenyltrazine, Oxybenzone, Octyltriazone, Octisalate, Padimate O, Bis-piperazine HAA299, (2-Ethoxyethyl (2Z)-2-cyano-2-[3-(3-methoxypropylamino) cyclohex-2-en-1-ylidene]acetate), Sulisobenzone, Drometrizole trisiloxane, Methylene bis-benzotriazolyl tetramethylbutylphenol, Benzylidene camphor sulfonic acid, 4-Methylbenzylidene Camphor, Polysilicone-15, Sulisobenzone sodium, Aminobenzoic acid, PEG-25 PABA, Menthyl anthranilate, Isopentyl-4-methoxycinnamate, Dioxibenzone, Cinoxate, camphor benzalkonium sulfate, and a mixture thereof.

13. The composition according to claim 1, wherein the UV filter is an inorganic compound selected from the group consisting of Zinc Oxide, Titanium Dioxide, Aluminum Stearate, Alumina, Hydrated Silica, Aluminum Hydroxide, a mixture thereof, and a surface modified oxide.

14. The composition according to claim 8, wherein the zeolites provide an SPF-boosting action to the composition of at least 20%.

15. The composition according to claim 1, further comprising a water-free phase selected from the group consisting of an oil, a wax, an emollient, an emulsifier, a silicone, a polymer, butter, a film former, a solubilizer, an organic or inorganic dispersant, a rheology modifier, fat, a solvent, a carrier, a stabilizer, and an organic or inorganic jellifying ingredient.

16. The composition according to claim 15 formulated as a stick, an oil or a gel.

17. The composition according to claim 1, wherein the composition is a homogenous oil-in-water emulsion.

18. The composition according to claim 1, further comprising:
at least one of a cosmetically acceptable emollient, carrier fluid, pigment, humectant, vitamin, antioxidant, emulsifier, co-emulsifier, hydrophilic or hydrophobic thickener, wax, butter, film former, silicone, surfactant, active agent, extract, fragrance, preservative, pH-adjusting agent, suspending agent, and chelating agent.

19. A method for protecting a keratinous surface from UV radiation, the method comprising: contacting the keratinous surface with the composition according to claim 1.
